(19) United States
(12) Patent Application Publication
(10) Pub. No.: US 2024/0293010 A1
(43) Pub. Date: Sep. 5, 2024

(54) INSERTION ASSISTING TOOL

(71) Applicant: FUJIFILM Corporation, Tokyo (JP)

(72) Inventors: Kyohei MORI, Kanagawa (JP); Makoto AIHARA, Kanagawa (JP); Nori MIYAGISHIMA, Kanagawa (JP)

(73) Assignee: FUJIFILM Corporation, Tokyo (JP)

(21) Appl. No.: 18/444,734

(22) Filed: Feb. 18, 2024

(30) Foreign Application Priority Data
Mar. 3, 2023 (JP) 2023-032999

(51) Int. Cl. A61B 1/00

(52) U.S. Cl. CPC A61B 1/00154; A61B 1/00066; A61B 1/00082

(57) ABSTRACT

An overtube includes an overtube body and a tubular gripping part that is connected to a proximal end side of the overtube body and that has an insertion port which communicates with an insertion pipe line. In cross-sectional view that is viewed from a direction orthogonal to a central axis direction of the gripping part of the overtube, at least a part of an outer peripheral surface of the gripping part has a shape formed by standardization.

### CROSS-REFERENCE TO RELATED APPLICATION

The present application claims priority under 35 U.S.C § 119(a) to Japanese Patent Application No. 2023-032999 filed on Mar. 3, 2023, which is hereby expressly incorporated by reference, in its entirety, into the present application.

### BACKGROUND OF THE INVENTION

### 1. Field of the Invention

The present invention relates to an insertion assisting tool that is inserted into a body together with an insertion part of an endoscope.

### 2. Description of the Related Art

A technique of inserting an insertion part of an endoscope into a digestive tract (also referred to as a luminal organ), such as a large intestine and a small intestine, and performing observation, diagnosis, and treatment of an inner wall surface of the digestive tract is performed in the medical field. The digestive tract, such as the large intestine and the small intestine, is bent in a complicated manner. Thus, it is difficult to transmit a force to a distal end of the insertion part simply by pushing the insertion part of the endoscope, and it is difficult to insert the endoscope into a deep portion.

Thus, in the related art, an insertion assisting tool (also referred to as an overtube) for guiding the insertion part of the endoscope into the body is used. For example, JP2008-136740A and WO2020/049913A disclose an insertion assisting tool provided with an inflatable and deflatable balloon at a tip part of the insertion assisting tool.

In such an insertion assisting tool with a balloon, a balloon control device can individually control the inflation and deflation of the balloon by supplying and sucking air into and from the balloon. Accordingly, the insertion part can be inserted into the deep portion of the digestive tract bent in a complicated manner by alternately inserting the insertion part and the insertion assisting tool while temporarily fixing the balloon of the insertion assisting tool to the digestive tract at a predetermined timing.

### SUMMARY OF THE INVENTION

Meanwhile, an insertion operation of alternately inserting the insertion part and the insertion assisting tool and a pulling-in operation of pulling the insertion part and the insertion assisting tool to a hand side are performed with the insertion assisting tool used in order to guide the insertion part of the endoscope. The operations are performed while an operator or an assistant grips a gripping part of the insertion assisting tool. For this reason, it is required for the gripping part of the insertion assisting tool to improve usability such as ease of gripping, finger contact, and ease of twisting for the operator or the assistant.

The present invention is devised in view of such circumstances, and an object thereof is to provide an insertion assisting tool in which usability of a gripping part is improved.

An insertion assisting tool according to an aspect of the present invention comprises a tube body that includes an insertion pipe line into which an endoscope insertion part is insertable and that is inserted into a body cavity and a tubular gripping part that is connected to a proximal end side of the tube body and that has an insertion port which communicates with the insertion pipe line, in which in cross-sectional view that is viewed from a direction orthogonal to an axial direction of the gripping part, at least a part of an outer peripheral surface of the gripping part has a shape formed by standardization.

According to the aspect of the present invention, in the outer peripheral surface of the gripping part, at least two surfaces facing each other in the direction orthogonal to the axial direction have the standardized shape.

According to the aspect of the present invention, in the outer peripheral surface of the gripping part, four surfaces facing each other in pairs in the direction orthogonal to the axial direction have the standardized shape.

According to the aspect of the present invention, the outer peripheral surface of the gripping part has a pair of side portions facing each other and a pair of arc portions connecting end parts of the pair of side portions to each other.

According to the aspect of the present invention, the side portion is an arc curve having a curvature radius larger than a curvature radius of the arc portion.

According to the aspect of the present invention, the side portion is a straight line.

According to the aspect of the present invention, a connecting portion between the side portion and the arc portion has a rounded shape without a corner portion.

According to the aspect of the present invention, the gripping part includes a first sleeve portion that is provided on a side of the tube body and a second sleeve portion that is provided on a side of the first sleeve portion opposite to the side of the tube body and that has an outer shape smaller than an outer shape of the first sleeve portion, and an outer peripheral surface of at least one of the first sleeve portion or the second sleeve portion has the pair of side portions and the pair of arc portions.

According to the aspect of the present invention, an outer peripheral surface of each of the first sleeve portion and the second sleeve portion has the pair of side portions and the pair of arc portions.

According to the aspect of the present invention, in cross-sectional view that is viewed from the direction orthogonal to the axial direction, the outer peripheral surface of the first sleeve portion and the outer peripheral surface of the second sleeve portion have shapes similar to each other.

According to the aspect of the present invention, the second sleeve portion has a length in the axial direction shorter than a length of the first sleeve portion in the axial direction.

According to the aspect of the present invention, the gripping part comprises an insertion port forming portion that is provided on a side of the second sleeve portion opposite to a side of the first sleeve portion, that has a funnel-like outer shape of which a diameter increases to the opposite side to the side of the first sleeve portion, and in which the insertion port is formed.

According to the aspect of the present invention, an outer peripheral surface of each of the first sleeve portion, the second sleeve portion, and the insertion port forming portion has the pair of side portions and the pair of arc portions.

According to the aspect of the present invention, in cross-sectional view that is viewed from the direction orthogonal to the axial direction, the outer peripheral surface of the first sleeve portion, the outer peripheral surface of the second sleeve portion, and the outer peripheral surface of the insertion port forming portion have shapes similar to each other.

According to the aspect of the present invention, a flange portion that has an outer shape larger than the first sleeve portion and the second sleeve portion, is further included between the first sleeve portion and the second sleeve portion.

According to the aspect of the present invention, the insertion port forming portion comprises a leakage prevention valve that prevents a liquid which has passed through the insertion pipe line from flowing out from the insertion port.

According to the aspect of the present invention, in cross-sectional view that is viewed from the direction orthogonal to the axial direction, the outer peripheral surface of the gripping part has a shape of which curvature continuously changes along a circumferential direction.

With the present invention, usability with respect to the gripping part of the insertion assisting tool can be improved.

### BRIEF DESCRIPTION OF THE DRAWINGS

FIG. 1 is a system configuration view of an endoscope device.

FIG. 2 is an enlarged perspective view of a tip part of an insertion part.

FIG. 3 is a side view of an overtube.

FIG. 4 is a perspective view of the overtube viewed from a side of a proximal end.

FIG. 5 is a cross-sectional view of a gripping part viewed from a direction orthogonal to an axial direction.

FIG. 6 is a cross-sectional view of a first sleeve portion and a second sleeve portion viewed from the direction orthogonal to the axial direction.

FIG. 7 is a view of an insertion port forming portion viewed from a proximal end side in the axial direction.

FIG. 8 is a view for describing an operation using the overtube.

FIG. 9 is a cross-sectional view of a gripping part according to a modification example viewed from the direction orthogonal to the axial direction.

FIG. 10 is a view for describing an assembly structure of the overtube.

FIG. 11 is a view for describing an assembly structure of a balloon air supply pipe and a liquid supply pipe.

FIG. 12 is a view for describing the assembly structure of the balloon air supply pipe and the liquid supply pipe.

FIG. 13 is a view for describing the assembly structure of the balloon air supply pipe and the liquid supply pipe.

### DESCRIPTION OF THE PREFERRED EMBODIMENTS

Hereinafter, an overtube according to a preferable embodiment of the present invention will be described in detail with reference to the accompanying drawings.

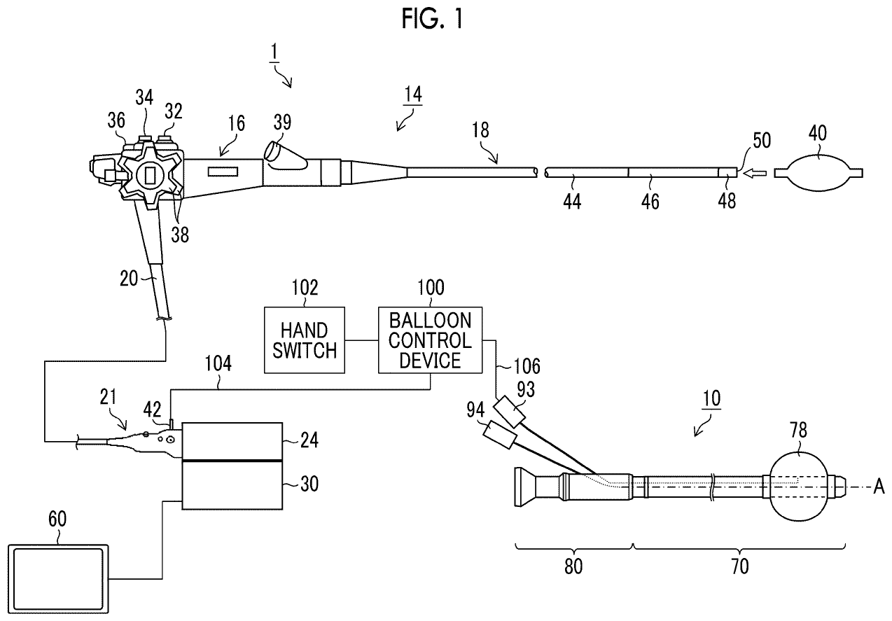

FIG. 1 is a system configuration view of an endoscope device 1 having an overtube 10 according to the embodiment of the present invention.

As shown in FIG. 1, the endoscope device 1 comprises an endoscope 14, the overtube 10, and a balloon control device 100. Although an endoscope for a lower digestive tract is given as an example of the endoscope 14, other endoscopes for an upperdigestive tract can also be applied.

### Endoscope

The endoscope 14 comprises a hand operating part 16 and an insertion part 18 which is installed consecutively to the hand operating part 16. A universal cable 20 is connected to the hand operating part 16. Although not shown, the universal cable 20 includes a signal cable, a light guide, and an air supply tube. A connector 21 connected to a light source device 24 is provided at a distal end of the universal cable 20. The light source device 24 is electrically connected to a processor 30. The light source device 24 and the connector 21 can transmit and receive a control signal and an image signal through optical communication. The light source device 24 transmits the control signal and the like transmitted and received via the connector 21 through optical communication to the processor 30. The light source device 24 wirelessly supplies power for driving the endoscope 14 via the connector 21. A monitor 60 is connected to the processor 30. The insertion part 18 is an example of an endoscope insertion part according to the embodiment of the present invention.

In addition, in the hand operating part 16, an air/water supply button 32, a suction button 34, and a shutter button 36 are arranged to be parallel to each other, and a pair of angle knobs 38 and 38 and a forceps insertion part 39 are provided. Further, the connector 21 is provided with a balloon air supply port 42 for supplying air to a balloon 40 to be described later or for sucking air from the balloon 40. The term “air” herein is a gas for inflating the balloon 40 (also including a balloon 78 to be described later), and a type (component) thereof is not particularly limited.

The insertion part 18 has a soft portion 44, a bendable part 46, and a tip part 48 from a proximal end side toward a distal end side of the insertion part 18. The bendable part 46 is remotely bent by rotationally moving the pair of angle knobs 38 and 38 provided in the hand operating part 16. Accordingly, a distal end surface 50 of the tip part 48 can be directed in a desired direction.

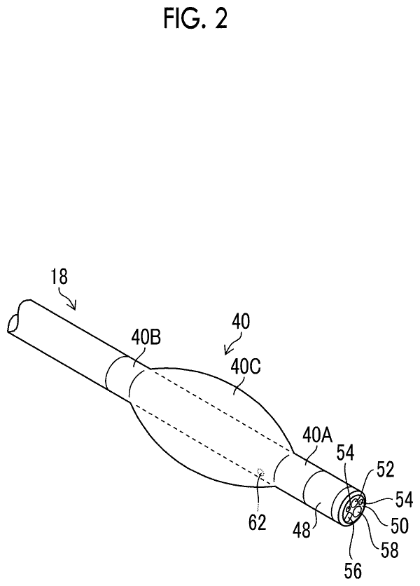

FIG. 2 is an enlarged perspective view showing the tip part 48 of the insertion part 18.

As shown in FIG. 2, the distal end surface 50 of the tip part 48 has an observation window 52, a pair of illumination windows 54 and 54, an air and water supply nozzle 56, and a forceps port 58. In the tip part 48, an imaging element (not shown) is provided behind the observation window 52. An observation image is formed on the imaging element and is photoelectrically converted. A signal cable (not shown) is connected to the imaging element, and the signal cable is inserted into the insertion part 18, the hand operating part 16, and the universal cable 20 shown in FIG. 1 to be projected up to the connector 21 and is connected to the light source device 24. An electric signal indicating the observation image photoelectrically converted by the imaging element is output from the light source device 24 to the processor 30 and then is output to the monitor 60 after the signal is appropriately processed. Accordingly, the observation image is displayed on the monitor 60.

An emission end of the light guide (not shown) is disposed behind each of the pair of illumination windows 54 and 54 in the tip part 48. An incidence end of each light guide is connected to the light source device 24 (see FIG. 1). Accordingly, an observed site is irradiated with illumination light, which is supplied from the light source device 24 to the incidence end of each light guide, from the emission end of each light guide via the pair of illumination windows 54 and 54.

An air supply and suction port 62 is provided in an outer peripheral surface of the tip part 48. The air supply and suction port 62 communicates with the balloon air supply port 42 via various types of tubes or the like (not shown) inserted from the inside of the insertion part 18 to the connector 21 (see FIG. 1). Accordingly, in a case where air is supplied from the balloon air supply port 42, the air is blown out from the air supply and suction port 62 to the outside via the tube or the like. In addition, in a case where the air is sucked from the balloon air supply port 42, the air is sucked from the air supply and suction port 62 via the tube or the like.

In addition, the balloon 40 formed of various types of elastic bodies or the like is attachably and detachably mounted on the tip part 48 of the insertion part 18. The balloon 40 comprises a bulging part 40C at a center thereof and mounting parts 40A and 40B on the distal end side and the proximal end side thereof. In a state where the air supply and suction port 62 is disposed on an inner side of the bulging part 40C of the balloon 40, each of the mounting parts 40A and 40B is fixed to the tip part 48 through a known method. The bulging part 40C of the balloon 40 configured as described above is inflated into a substantially spherical shape by blowing air from the air supply and suction port 62, and the bulging part 40C is deflated by sucking air from the air supply and suction port 62.

### Overtube

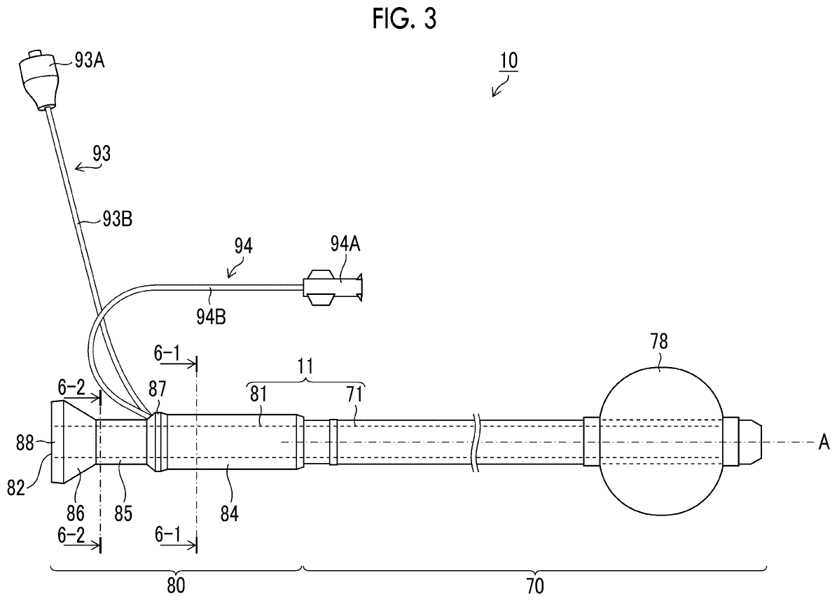

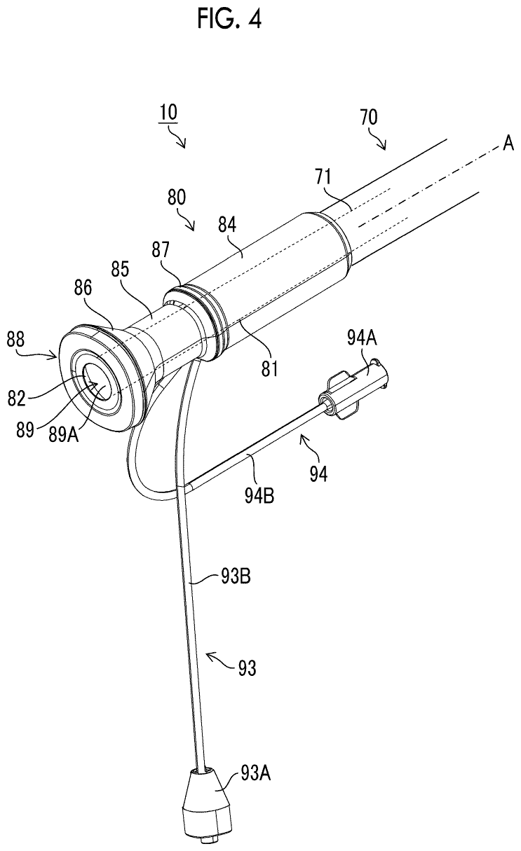

FIG. 3 is a side view of the overtube 10. In addition, FIG. 4 is an enlarged perspective view of the overtube 10 viewed from the proximal end side.

As shown in FIGS. 3 and 4, the overtube 10 has an overtube body 70. The overtube body 70 is formed of various flexible materials or the like in a tubular shape. The outer shape of the overtube body 70 has a substantially fixed size along a central axis A direction. Inside the overtube body 70, a first insertion pipe line 71 is formed along the central axis A direction. An inner peripheral surface of the first insertion pipe line 71 of the overtube body 70 has an inner diameter slightly larger than the outer diameter of the insertion part 18.

A tubular gripping part 80 to be gripped by an operator is connected to the proximal end side of the overtube body 70. Inside the gripping part 80, a second insertion pipe line 81 is formed along the central axis A direction. An insertion port 82 that communicates with the second insertion pipe line 81 is formed on the proximal end side of the gripping part 80. By connecting the overtube body 70 and the gripping part 80 to each other, the first insertion pipe line 71 and the second insertion pipe line 81 communicate with each other, forming an insertion pipe line 11. The insertion port 82 communicates with the insertion pipe line 11 of the overtube 10. The insertion part 18 is movable along the central axis A direction inside the insertion pipe line 11. In the present specification, the “distal end” or the “distal end side” refers to a side in a direction of being inserted into a body cavity, and a “proximal end” or the “proximal end side” refers to an opposite side to the “distal end” or the “distal end side”. The overtube 10 is an example of an insertion assisting tool according to the embodiment of the present invention. The overtube body 70 is an example of a tube body according to the embodiment of the present invention. The gripping part 80 is an example of a gripping part according to the embodiment of the present invention. The insertion pipe line 11 is an example of an insertion pipe line according to the embodiment of the present invention. The insertion port 82 is an example of an insertion port according to the embodiment of the present invention. The central axis A direction is an example of an axial direction according to the embodiment of the present invention.

The balloon 78 formed of various types of elastic bodies is mounted on the outer peripheral surface of the overtube body 70 on the distal end side. In addition, an air supply and discharge pipe line (not shown) is formed in the overtube body 70 along the central axis A direction between the outer peripheral surface and an inner peripheral surface. At an outer peripheral surface of the balloon 78 positioned on the inner side, the air supply and discharge pipe line is open as an air supply and suction port (not shown) with respect to the balloon 78.

A balloon air supply pipe 93 that communicates with the air supply and discharge pipe line is provided at an outer peripheral surface of the gripping part 80. The balloon air supply pipe 93 is composed of a balloon air supply port 93A and a balloon air supply tube 93B.

The balloon air supply port 93A is connected to the balloon control device 100 via a tube 106 (see FIG. 1). In a case where the balloon control device 100 is driven to supply air to the balloon air supply port 93A, the air is blown from the air supply and suction port via the balloon air supply tube 93B and the air supply and discharge pipe line of the overtube body 70. Accordingly, the balloon 78 is inflated. In addition, in a case where the air is sucked by the balloon control device 100, the air in the balloon 78 is sucked from the air supply and suction port via the air supply and discharge pipe line. Accordingly, the balloon 78 is deflated.

As shown in FIG. 1, the balloon control device 100 is connected to the balloon air supply port 42 of the endoscope 14 via a tube 104 and is connected to the balloon air supply pipe 93 of the overtube body 70 via the tube 106. In addition, the balloon control device 100 is connected to a hand switch 102. The balloon control device 100 supplies air to each of the balloons 40 and 78 or sucks the air in each of the balloons 40 and 78 in accordance with a control signal from the hand switch 102. Accordingly, each of the balloons 40 and 78 is individually inflated or deflated.

A liquid supply pipe 94 for supplying a lubricant, such as water, to the insertion pipe line 11 of the overtube 10 is provided at the outer peripheral surface of the gripping part 80. The liquid supply pipe 94 is composed of a liquid supply port 94A and a liquid supply tube 94B. A lubricant supply unit (not shown) such as a syringe is connected to the liquid supply port 94A. The lubricant supplied from the liquid supply pipe 94 reduces friction between an inner peripheral surface of the insertion pipe line 11 of the overtube 10 and an outer peripheral surface of the insertion part 18 (of the endoscope 14) inserted into the insertion pipe line 11.

### Gripping Part

The gripping part 80 is formed of various types of hard materials in a tubular shape. As shown in FIGS. 3 and 4, the gripping part 80 comprises a first sleeve portion 84, a second sleeve portion 85, and an insertion port forming portion 86 from the distal end side toward the proximal end side in this order. The first sleeve portion 84 has a tubular shape. The first sleeve portion 84 is positioned on the proximal end side of the overtube body 70. The first sleeve portion 84 is an example of a first sleeve portion according to the embodiment of the present invention.

The second sleeve portion 85 has a tubular shape and is provided on the proximal end side (the opposite side of the overtube body 70) of the first sleeve portion 84. The second sleeve portion 85 has an outer shape larger than the first sleeve portion 84. The second sleeve portion 85 is an example of a second sleeve portion according to the embodiment of the present invention.

The gripping part 80 has, between the first sleeve portion 84 and the second sleeve portion 85, a flange portion 87 that has an outer shape larger than the first sleeve portion 84 and the second sleeve portion 85. The flange portion 87 is an example of a flange portion according to the embodiment of the present invention.

The flange portion 87 is a portion protruding from outer peripheral surfaces of the first sleeve portion 84 and the second sleeve portion 85 toward an outer side in a radial direction and is formed in a disk shape over the entire periphery of the gripping part 80.

The insertion port forming portion 86 is provided on the proximal end side (an opposite side to a side of the first sleeve portion 84) of the second sleeve portion 85. The insertion port forming portion 86 has a funnel-like outer shape of which a diameter increases from the distal end side (the side of the first sleeve portion 84) toward the proximal end side (the opposite side to the side of the first sleeve portion 84) of the insertion port forming portion 86. The insertion port 82 is formed on the proximal end side of the insertion port forming portion 86. The insertion port forming portion 86 is an example of an insertion port forming portion according to the embodiment of the present invention.

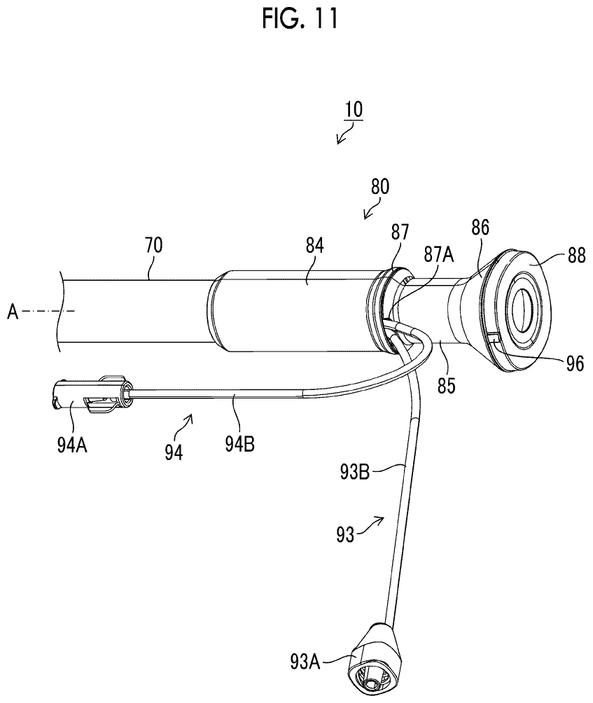

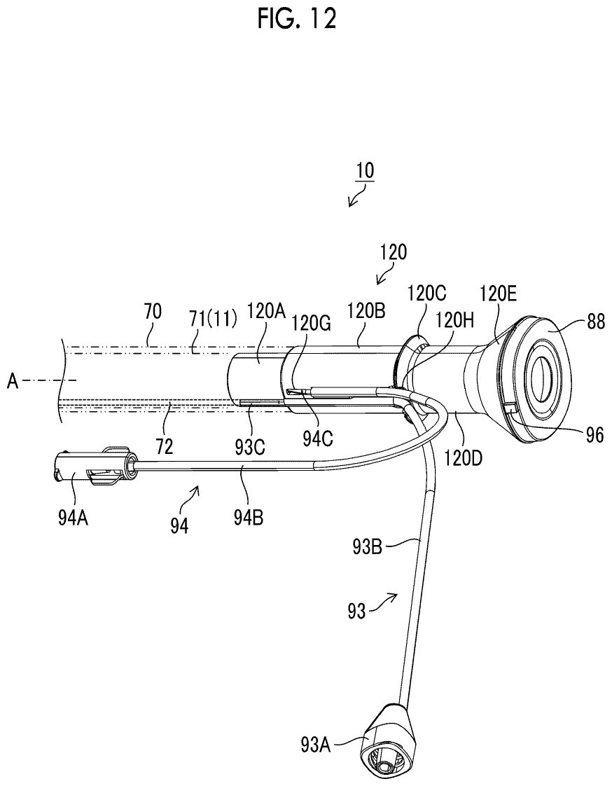

The proximal end side of the insertion port forming portion 86 is composed of a proximal end cap 88. The proximal end cap 88 has an outer shape that is the same shape of a funnel-like maximum diameter portion of the insertion port forming portion 86. An opening that is the insertion port 82 is formed in the proximal end cap 88. A protrusion 96 that is a marker for being attached to the insertion port forming portion 86 is formed on an outer peripheral surface of the proximal end cap 88 (see FIGS. 11 and 12 to be described later). In addition, a leakage prevention valve 89 is provided inside the proximal end cap 88. The leakage prevention valve 89 is a valve member provided in order to prevent a body fluid (liquid) flowed from the distal end side of the insertion pipe line 11 to the inside from flowing out from the insertion port 82 in a case of being inserted into a body in a state where the insertion part 18 is inserted into the insertion pipe line 11 of the overtube 10. The leakage prevention valve 89 consists of an elastic member, and an opening 89A for inserting the insertion part 18 into a central portion thereof is formed. The leakage prevention valve 89 prevents a body fluid that has passed through the insertion pipe line 11 from flowing out by being closely attached to the outer peripheral surface of the insertion part 18 in a case where the insertion part 18 is inserted into the insertion pipe line 11. The leakage prevention valve 89 is an example of a leakage prevention valve according to the embodiment of the present invention.

### Outer Peripheral Surface Shape of Gripping Part

Next, the shape of an outer peripheral surface 90 of the gripping part 80 that is a characteristic portion according to the embodiment of the present invention will be described. The gripping part 80 according to the embodiment is composed of the first sleeve portion 84, the second sleeve portion 85, and the insertion port forming portion 86, and shapes of outer peripheral surfaces of respective portions are shapes similar to each other. Hereinafter, in a case of describing common matters of the shape of the outer peripheral surface of each portion (the first sleeve portion 84, the second sleeve portion 85, and the insertion port forming portion 86) configuring the gripping part 80, the outer peripheral surface 90 of the gripping part 80 will be used as a generic term.

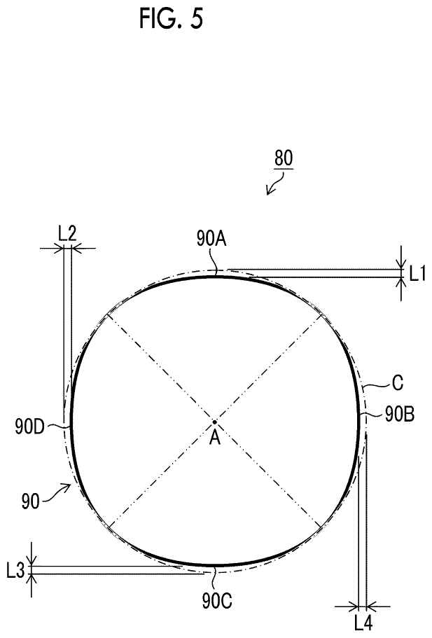

FIG. 5 is a view for describing the shape of the outer peripheral surface 90 of the gripping part 80 and is a cross-sectional view in a case where the gripping part 80 is viewed from a direction orthogonal to the central axis A direction. In FIG. 5, a solid line indicates the outer peripheral surface 90 of the gripping part 80. In addition, a two-dot chain line indicates a circumscribed circle C circumscribing the outer peripheral surface 90 of the gripping part 80.

As shown in FIG. 5, in a case where the outer peripheral surface 90 of the gripping part 80 is divided into four regions at equal intervals (for each 90 degrees) in a circumferential direction of which a center is a central axis A, a first surface 90A, a second surface 90B, a third surface 90C, and a fourth surface 90D that are four surfaces in respective regions have convex shapes toward the outer side and are formed at positions slightly more shifted to the central axis A side than the circumscribed circle C is. In other words, the outer peripheral surface 90 of the gripping part 80 is obtained by standardizing each of the four regions (surfaces) divided in the circumferential direction with respect to the circumscribed circle C.

Specifically, as shown in FIG. 5, a part (a central portion of each surface in FIG. 5) of each of the first surface 90A, the second surface 90B, the third surface 90C, and the fourth surface 90D configuring the outer peripheral surface 90 is positioned on a side close to the central axis A by distances L1, L2, L3, and L4 compared to the circumscribed circle C. The term “standardizing” in the present specification means a state where at least a part (the four surfaces in the present example) of the outer peripheral surface 90 of the gripping part 80 is at a position more shifted to the central axis A side than the circumscribed circle C circumscribing the outer peripheral surface 90 of the gripping part 80 is (however, excluding a state of being recessed to the central axis A side in a concave shape).

In the example shown in FIG. 5, the outer peripheral surface 90 of the gripping part 80 is configured in a shape in which the four surfaces facing each other in pairs (a pair of the first surface 90A and the third surface 90C and a pair of the second surface 90B and the fourth surface 90D) in the direction orthogonal to the central axis A direction are standardized.

As the outer peripheral surface 90 of the gripping part 80 comprises each of the standardized surfaces (the first surface 90A, the second surface 90B, the third surface 90C, and the fourth surface 90D) which are standardized as described above, an area where the outer peripheral surface 90 and a finger come into contact with each other increases in a case where the operator or an assistant performs work. In addition, in the outer peripheral surface 90 of the gripping part 80, a boundary portion (connecting portion) that connects standardized surfaces adjacent to each other has a curvature radius smaller than those of the standardized surfaces, and the finger easily hangs onto the outer peripheral surface 90.

In the outer peripheral surface 90 of the gripping part 80, the distances L1, L2, L3, and L4 between each standardized surface and the circumscribed circle C are the same distance. Some of the distances L1, L2, L3, and L4 may be distances different from each other, or all the distances may be different from each other. For example, the distances L1 and L3 of the first surface 90A and the third surface 90C facing each other are equal to each other (L1=L3), the distances L2 and L4 of the second surface 90B and the fourth surface 90D facing each other are equal to each other (L2=L4), and distances of surfaces in a pair may be different from each other (L1≠L2) in some cases.

### First Sleeve Portion, Second Sleeve Portion, and Insertion Port Forming Portion

The shapes of the outer peripheral surfaces of the first sleeve portion 84, the second sleeve portion 85, and the insertion port forming portion 86 will be described.

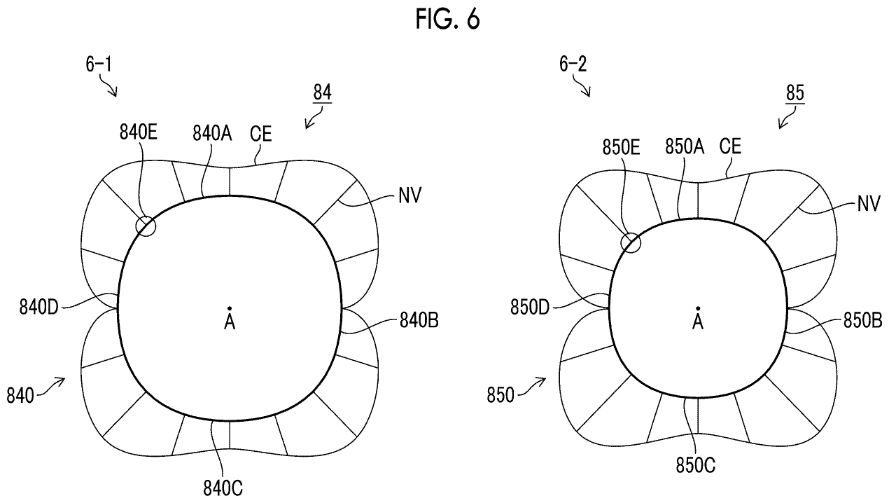

6-1 of FIG. 6 is a cross-sectional view (a cross-sectional view taken along line 6-1 of FIG. 3) of the first sleeve portion 84 viewed from the direction orthogonal to the central axis A direction. 6-2 of FIG. 6 is a cross-sectional view (a cross-sectional view taken along line 6-2 of FIG. 3) of the second sleeve portion 85 viewed from the direction orthogonal to the central axis A direction.

In 6-1 of FIG. 6, the shape of a first outer peripheral surface 840 of the first sleeve portion 84 is shown by a normal vector NV and a curvature envelope CE. The normal vector NV is a vector orthogonal to the first outer peripheral surface 840. The length of the normal vector NV indicates the magnitude of curvature at a point where the normal vector NV is derived. That is, in a case where the length of the normal vector NV is long, curvature at that point is large (the curvature radius is small), and in a case where the length of the normal vector NV is short, curvature at that point is small (the curvature radius is large). The curvature envelope CE is a line diagram showing a relative magnitude of curvature of the first outer peripheral surface 840 and shows a change in the curvature of the first outer peripheral surface 840.

As shown in 6-1 of FIG. 6, the first outer peripheral surface 840 has a shape that is line-symmetrical with respect to a straight line passing through the central axis A and is configured in a shape of which curvature changes continuously along the circumferential direction. The first outer peripheral surface 840 is composed of four standardized surfaces and comprises a pair of side portions 840A and 840C facing each other and a pair of arc portions 840B and 840D facing each other. The pair of arc portions 840B and 840D connect end parts of the pair of side portions 840A and 840C to each other.

In 6-2 of FIG. 6, as in 6-1 of FIG. 6, the shape of a second outer peripheral surface 850 of the second sleeve portion 85 is shown by the normal vector NV and the curvature envelope CE. The second outer peripheral surface 850 of the second sleeve portion 85 is configured in a shape similar to the first outer peripheral surface 840 of the first sleeve portion 84 described above and has a shape relatively smaller than the first outer peripheral surface 840.

As shown in 6-2 of FIG. 6, the second outer peripheral surface 850 has a shape that is line-symmetrical with respect to a straight line passing through the central axis A and is configured in a shape of which curvature changes continuously along the circumferential direction. The second outer peripheral surface 850 is composed of four standardized surfaces and comprises a pair of side portions 850A and 850C facing each other and a pair of arc portions 850B and 850D facing each other. The pair of arc portions 850B and 850D connect end parts of the pair of side portions 850A and 850C to each other.

The first outer peripheral surface 840 of the first sleeve portion 84 is an example of an outer peripheral surface of the first sleeve portion according to the embodiment of the present invention. The second outer peripheral surface 850 of the second sleeve portion 85 is an example of an outer peripheral surface of the second sleeve portion according to the embodiment of the present invention. The pair of side portions 840A and 840C facing each other and the pair of side portions 850A and 850C facing each other are examples of the pair of side portions facing each other according to the embodiment of the present invention. The pair of arc portions 840B and 840D and the pair of arc portions 850B and 850D are examples of a pair of arc portions according to the embodiment of the present invention.

The curvature radius of the first outer peripheral surface 840 (the pair of arc-shaped side portions 840A and 840C and the pair of arc portions 840B and 840D) of the first sleeve portion 84 is preferably within a range of 6.0 mm or more to 15.0 mm or less. The curvature radius of the second outer peripheral surface 850 (the pair of arc-shaped side portions 850A and 850C and the pair of arc portions 850B and 850D) of the second sleeve portion 85 is preferably within a range of 4.0 mm or more to 13.0 mm or less.

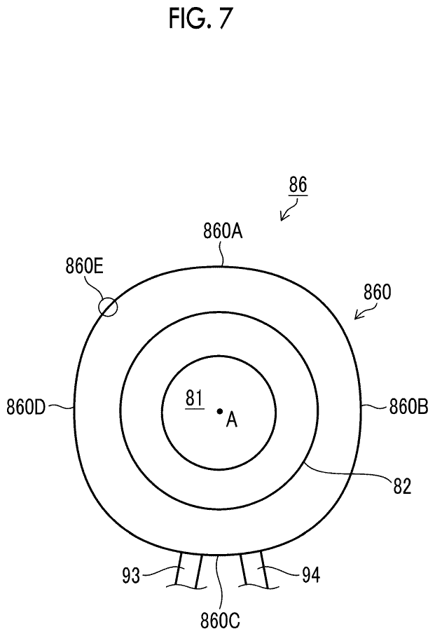

FIG. 7 is a view of the insertion port forming portion 86 viewed from the proximal end side in the central axis A direction. As described above, a proximal end part of the insertion port forming portion 86 is composed of the proximal end cap 88 and is configured in a shape similar to the first outer peripheral surface 840 of the first sleeve portion 84 and the second outer peripheral surface 850 of the second sleeve portion 85. An outer peripheral surface 860 of the insertion port forming portion 86 has a shape that is line-symmetrical with respect to a straight line passing through the central axis A and is configured in a shape of which curvature changes continuously along the circumferential direction. The outer peripheral surface 860 of the insertion port forming portion 86 is composed of four standardized surfaces and comprises a pair of side portions 860A and 860C and a pair of arc portions 860B and 860D. The pair of side portions 860A and 860C are examples of a pair of side portions according to the embodiment of the present invention. The pair of arc portions 860B and 860D are examples of the pair of arc portions according to the embodiment of the present invention.

In the gripping part 80 according to the embodiment, as shown in 6-1 of FIG. 6, a connecting portion 840E (shown by ∘) of the first outer peripheral surface 840 of the first sleeve portion 84, which is a boundary portion between the side portion (the side portions 840A and 840C) and the arc portion (the arc portions 840B and 840D), is a portion having curvature larger than those of the side portion and the arc portion and has a rounded shape without a corner portion.

In addition, as shown in 6-2 of FIG. 6, similarly in the second outer peripheral surface 850 of the second sleeve portion 85, a connecting portion 850E, which is a boundary portion between the side portion (the side portions 850A and 850C) and the arc portion (the arc portions 850B and 850D), is a portion having curvature larger than those of the side portion and the arc portion and has a rounded shape without a corner portion.

In addition, as shown in FIG. 7, similarly in the outer peripheral surface 860 of the insertion port forming portion 86, a connecting portion 860E, which is a boundary portion between the side portion (the side portions 860A and 860C) and the arc portion (the arc portions 860B and 860D), is a portion having curvature larger than those of the side portion and the arc portion and has a rounded shape without a corner portion. The connecting portions 840E, 850E, and 860E are examples of a connecting portion according to the embodiment of the present invention.

The following range can be preferably applied to the size and length of each of outer shapes of the first sleeve portion 84, the second sleeve portion 85, the flange portion 87, and the insertion port forming portion 86.

The outer shape of the first sleeve portion 84 is larger than the outer shape of the overtube body 70 and has a substantially fixed size along the central axis A direction. Specifically, the maximum diameter (the diameter of the circumscribed circle) of the outer peripheral surface of the first sleeve portion 84 is larger than the outer diameter of the outer peripheral surface (cylindrical surface) of the overtube body 70, is, for example, 10 mm or more and 40 mm or less, and is preferably 15 mm or more and 25 mm or less (more preferably 17 mm or more and 20 mm or less). In addition, the length of the first sleeve portion 84 in the central axis A direction is, for example, 20 mm or more and 100 mm or less and is preferably 35 mm or more and 60 mm or less (more preferably 40 mm or more and 50 mm or less).

The outer shape of the second sleeve portion 85 is smaller than the outer shape of the first sleeve portion 84 and has a substantially fixed size along the central axis A direction. In addition, the second sleeve portion 85 has a length in the central axis A direction shorter than that of the first sleeve portion 84. Specifically, the maximum diameter (the diameter of the circumscribed circle) of the outer peripheral surface of the second sleeve portion 85 is smaller than the maximum diameter (the diameter of the circumscribed circle) of the outer peripheral surface of the first sleeve portion 84, is, for example, 8 mm or more and 30 mm or less, and preferably 10 mm or more and 20 mm or less (more preferably 12 mm or more and 16 mm or less). In addition, the length of the second sleeve portion 85 in the central axis A direction is, for example, 5 mm or more and 50 mm or less and is preferably 8 mm or more and 30 mm or less (more preferably 10 mm or more and 20 mm or less).

The outer diameter of a circular outer edge portion of the flange portion 87 is larger than maximum diameters (the diameters of the circumscribed circles) of the first sleeve portion 84 and the second sleeve portion 85, is, for example, 12 mm or more and 50 mm or less, and is preferably 14 mm or more and 30 mm or less (more preferably 16 mm or more and 25 mm or less).

The outer shape of the proximal end part (proximal end cap 88) of the insertion port forming portion 86 is larger than the maximum diameters (the diameters of the circumscribed circles) of the first sleeve portion 84 and the second sleeve portion 85 and is preferably larger than the outer diameter of the circular outer edge portion of the flange portion 87. Specifically, the maximum diameter (the diameter of the circumscribed circle) of the outer peripheral surface of the proximal end part of the insertion port forming portion 86 is larger than the maximum diameters (the diameters of the circumscribed circles) of the outer peripheral surfaces of the first sleeve portion 84 and the second sleeve portion 85, is, for example, 15 mm or more and 50 mm or less, and is preferably 18 mm or more and 40 mm or less (more preferably 20 mm or more and 30 mm or less). In addition, the length of the insertion port forming portion 86 in the central axis A direction is, for example, 5 mm or more and 30 mm or less and is preferably 7 mm or more and 20 mm or less (more preferably 8 mm or more and 12 mm or less).

Although a case where each of the side portions 840A and 840C of the first outer peripheral surface 840, the side portions 850A and 850C of the second outer peripheral surface 850, and the side portions 860A and 860C of the outer peripheral surface 860 is composed of an arc curve has been given as an example in the gripping part 80 according to the embodiment, the invention is not limited to the configuration, and some or all of them may be composed of a straight line.

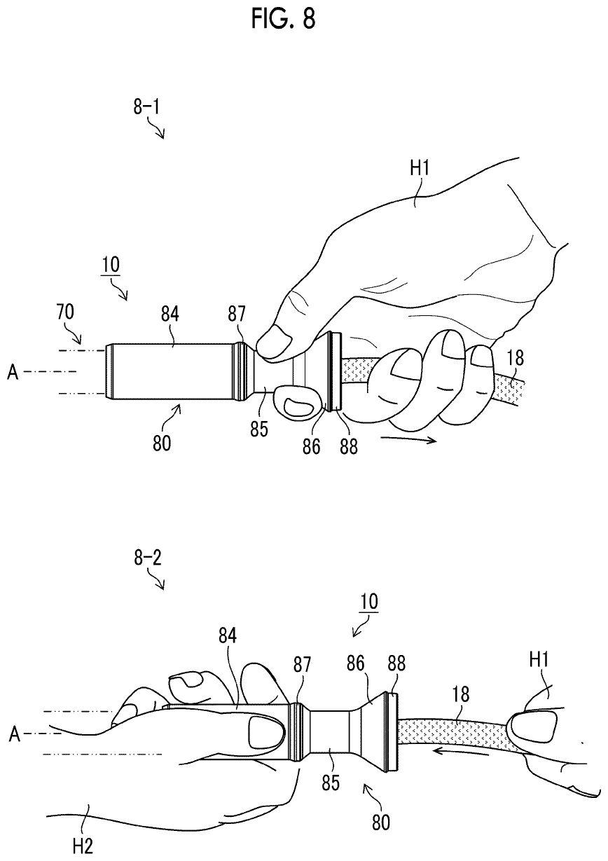

FIG. 8 is a view for describing an operation performed by the operator or the assistant gripping the gripping part 80 of the overtube 10. 8-1 of FIG. 8 shows a state where an operator H1 performs a pulling-in operation of the overtube 10 to a hand side. As a premise of the pulling-in operation, the balloon 78 of the overtube body 70 and the balloon 40 of the insertion part 18 are inflated and are fixed in the intestine.

The operator H1 performs an operation of pulling both the overtube body 70 and the insertion part 18 in an arrow direction and folding the intestine in a state where the balloons 40 and 78 are inflated. The operator H1 grips the second sleeve portion 85 with fingers (the thumb and the index finger) in a case of the pulling-in operation. Then, the operator H1 grips and pulls the insertion part 18 together with other fingers and the palm. Since the second outer peripheral surface 850 (not shown) of the second sleeve portion 85 is standardized as described above, a contact area with the fingers increases. In addition, in the second outer peripheral surface 850, a boundary portion (connecting portion) that connects standardized surfaces adjacent to each other has curvature larger (curvature radius smaller) than those of the standardized surfaces, and the fingers easily hang onto the second outer peripheral surface 850. In addition, since the insertion port forming portion 86 is formed on the proximal end side of the second sleeve portion 85, the fingers of the operator easily hang onto an increased diameter portion of the insertion port forming portion 86, and it becomes easy for the operator H1 to pull the overtube 10 and the insertion part 18.

8-2 of FIG. 8 shows a state where the operator H1 and an assistant H2 perform an insertion operation of inserting the insertion part 18 into the overtube 10. As a premise of the insertion operation, the overtube 10 is fixed in the intestine by the inflated balloon 78.

In a case of a pushing operation, the operator H1 grips the insertion part 18 in a state where the balloon is deflated with the fingers and the palm. The operator H1 pushes the insertion part 18 in the arrow direction. The assistant H2 grips the first sleeve portion 84 with the fingers (the thumb, the index finger, and the middle finger) such that the overtube 10 does not move. Since the first outer peripheral surface 840 (not shown) of the first sleeve portion 84 is standardized as described above, a contact area with the fingers increases, and the assistant H2 can firmly grip the gripping part 80. In this case, the fingers of the assistant H2 come into contact with the flange portion 87, the gripping part 80 becomes difficult to slip out of the palm of the assistant H2, and thereby a gripped state is stable.

As shown in FIG. 8, since the assistant H2 grips the first sleeve portion 84 with the fingers (the thumb, the index finger, and the middle finger), an outer shape that is long in the central axis A direction and that is large is preferable. On the other hand, since the operator grips the second sleeve portion 85 with the fingers (the thumb and the index finger), an outer shape that is short in the central axis A direction and that is small is preferable.

With the overtube 10 according to the embodiment, which is configured as described above, since the outer peripheral surface 90 (the first outer peripheral surface 840, the second outer peripheral surface 850, and the outer peripheral surface 860) of the gripping part 80 has a standardized surface, a contact area with the fingers increases in a case where the operator or the assistant grips the gripping part 80, and the fingers easily hang. Therefore, ease of gripping, finger contact, and ease of twisting with respect to the gripping part 80 for the operator or the assistant improve, and usability of the gripping part 80 improves.

### Modification Example of Outer Peripheral Surface of Gripping Part

Although a configuration where the outer peripheral surface 90 (the first outer peripheral surface 840, the second outer peripheral surface 850, and the outer peripheral surface 860) of the gripping part 80 has four standardized surfaces has been described in the embodiment, the invention is not limited thereto, and at least a part of the outer peripheral surface 90 may be standardized.

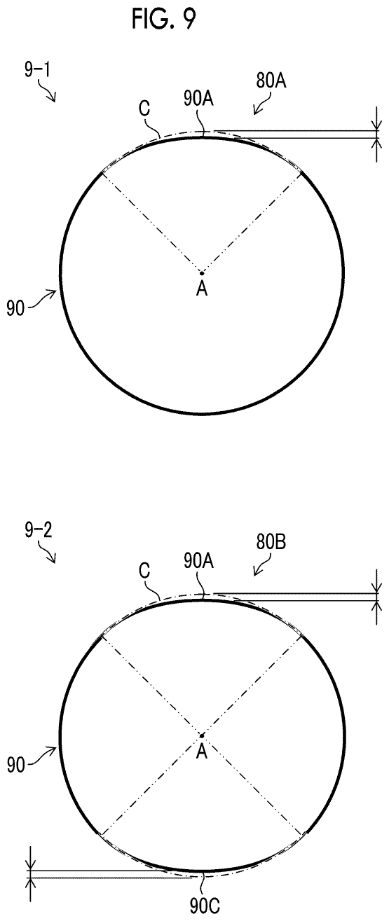

FIG. 9 is a cross-sectional view of a gripping part according to a modification example viewed from a direction orthogonal to the axial direction. In the outer peripheral surface 90 of a gripping part 80A according to the modification example shown in 9-1 of FIG. 9, only the first surface 90A is composed of a standardized surface. A surface of the outer peripheral surface 90 excluding the first surface 90A is not standardized and matches the circumscribed circle C.

In the outer peripheral surface 90 of a gripping part 80B according to a modification example shown in 9-2 of FIG. 9, the first surface 90A and the third surface 90C facing each other are composed of standardized surfaces. A surface of the outer peripheral surface 90 excluding the first surface 90A and the third surface 90C is not standardized and matches the circumscribed circle C.

Even with a configuration where at least a part of the outer peripheral surface 90 of the gripping part 80B is standardized as in the modification examples, ease of gripping, finger contact, and ease of twisting with respect to the gripping part 80 or the like for the operator or the assistant improve, and usability of the gripping part 80 improves.

In addition, although a configuration where the first outer peripheral surface 840, the second outer peripheral surface 850, and the outer peripheral surface 860 corresponding to the outer peripheral surface 90 of the gripping part 80 have shapes similar to each other has been described in the embodiment as one preferable aspect, the invention is not limited thereto, and some or all of them may have dissimilar shapes.

### Assembly Structure of Overtube

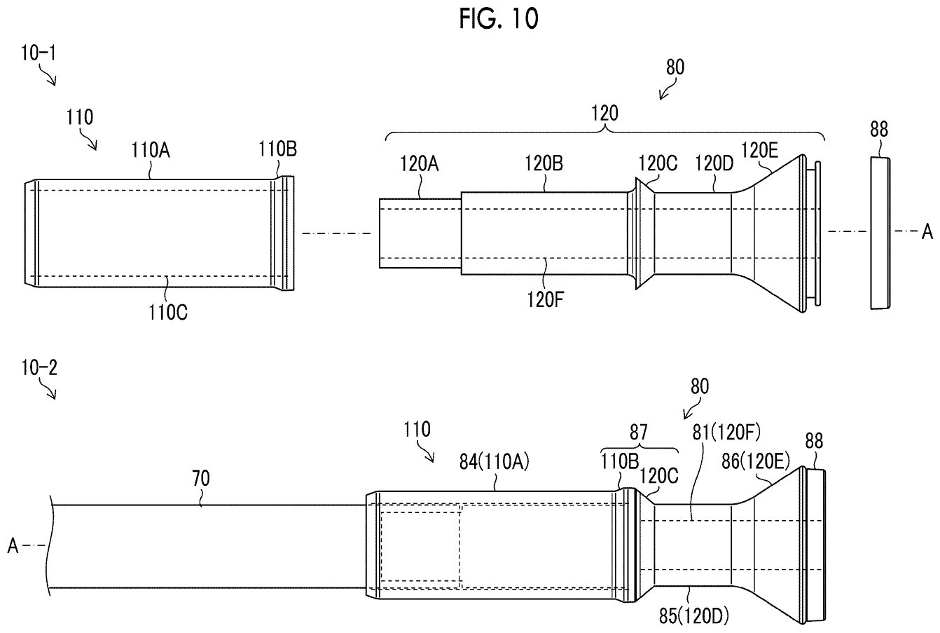

FIG. 10 is a view for describing an assembly structure of the overtube 10. In FIG. 10, 10-1 is a view showing a state before assembly of the gripping part 80, and 10-2 is a view showing a state after assembly of the gripping part 80.

As shown in FIG. 10, the gripping part 80 comprises a first member 110, a second member 120, and the proximal end cap 88. The first member 110 comprises a first body part 110A and a first flange portion 110B from the distal end side toward the proximal end side in this order. The first member 110 is composed of the first body part 110A and the first flange portion 110B which are integrally molded. The first member 110 has a sleeve shape in which a fitting passage 110C penetrating in the central axis A direction is formed.

The first body part 110A has a tubular outer shape. Since the first body part 110A becomes the first sleeve portion 84 after assembly, at least a part of the outer peripheral surface has a shape (standardized shape) shown in 6-1 of FIG. 6 in cross-sectional view that is viewed from the direction orthogonal to the central axis A direction.

The first flange portion 110B is provided on the proximal end side of the first body part 110A. The first flange portion 110B has a shape of which a diameter gradually increases toward the proximal end side.

The second member 120 has a first fitted portion 120A, a second fitted portion 120B, a second flange portion 120C, a second body part 120D, and a diameter increased portion 120E from the distal end side toward the proximal end side in this order. The second member 120 is composed of the first fitted portion 120A, the second fitted portion 120B, the second flange portion 120C, the second body part 120D, and the diameter increased portion 120E which are integrally molded. A penetration passage 120F that penetrates in the central axis A direction is formed in the second member 120.

The first fitted portion 120A has a tubular shape. The first fitted portion 120A is a portion fitted to the first insertion pipe line 71 of the overtube body 70. Due to the fitting, the gripping part 80 and the overtube body 70 are connected to each other.

The second fitted portion 120B is disposed on the proximal end side of the first fitted portion 120A and has a tubular shape. The second fitted portion 120B has an outer shape larger than the first fitted portion 120A. The second fitted portion 120B is a portion fitted to the fitting passage 110C of the first member 110. Due to the fitting, the first member 110 and the second member 120 are connected to each other.

The second flange portion 120C is disposed on the proximal end side of the second fitted portion 120B, protrudes from the second fitted portion 120B in an outer diameter direction, and has a shape of which a diameter gradually decreases toward the proximal end. After assembly of the gripping part 80, the first flange portion 110B and the second flange portion 120C become the flange portion 87.

The second body part 120D is disposed on the proximal end side of the second flange portion 120C and has a tubular shape. The outer shape of the second body part 120D is smaller than the outer shape of the first body part 110A of the first member 110. The second body part 120D is a portion that becomes the second sleeve portion 85 after assembly of the gripping part 80. That is, an outer peripheral surface of the second body part 120D has a shape (standardized shape) shown in 6-2 of FIG. 6.

The diameter increased portion 120E is disposed on the proximal end side of the second body part 120D and has a funnel-like outer shape of which a diameter gradually increases toward the proximal end. The diameter increased portion 120E is a portion that becomes the insertion port forming portion 86 after assembly of the gripping part 80. That is, an outer peripheral surface of the diameter increased portion 120E has a shape (standardized shape) shown in FIG. 7. A cylindrical proximal end cap mounting part is provided on the proximal end side of the diameter increased portion 120E, and the proximal end cap 88 is attached to an outer peripheral portion of the proximal end cap mounting part.

### Assembly Structure of Balloon Air Supply Pipe and Liquid Supply Pipe

An assembly structure of the balloon air supply pipe 93 and the liquid supply pipe 94 will be described based on FIGS. 11 to 13. As shown in FIG. 11, the balloon air supply pipe 93 and the liquid supply pipe 94 are derived from the flange portion 87 of the gripping part 80. Specifically, the balloon air supply tube 93B and the liquid supply tube 94B are derived from an opening 87A formed in a surface of the flange portion 87 on the proximal end side to the outside. Since the balloon air supply tube 93B and the liquid supply tube 94B are accommodated in the opening 87A, misregistration of the balloon air supply tube 93B and the liquid supply tube 94B from the gripping part 80 is suppressed.

FIG. 12 shows a state where the first member 110 (not shown) is removed from the overtube 10 of FIG. 11. As shown in FIG. 12, a through-hole 120G that communicates with the insertion pipe line 11 of the overtube 10 is formed in an outer peripheral surface of the second fitted portion 120B of the second member 120. A metal pipe 94C is attached to an end part of the liquid supply tube 94B on the proximal end side (the opposite side to the liquid supply port 94A). The metal pipe 94C is inserted into the through-hole 120G and communicates with the insertion pipe line 11. The metal pipe 94C has a shape bent to a side of the insertion pipe line 11. A lubricant such as water is supplied to the insertion pipe line 11 via the liquid supply port 94A, the liquid supply tube 94B, and the metal pipe 94C.

A metal pipe 93C is attached to an end part of the balloon air supply tube 93B on the proximal end side (the opposite side to the balloon air supply port 93A). The metal pipe 93C is inserted into an air supply and discharge pipe line 72 provided at the overtube body 70. The metal pipe 93C has a substantially linear shape. Air is supplied or discharged with respect to the balloon 78 via the balloon air supply port 93A, the balloon air supply tube 93B, the metal pipe 94C, and the air supply and discharge pipe line 72.

A groove 120H through which the balloon air supply tube 93B and the liquid supply tube 94B pass is formed in the second flange portion 120C of the second member 120. The opening 87A (see FIG. 11) is formed by the groove 120H of the second member 120 and the first flange portion 110B of the first member 110.

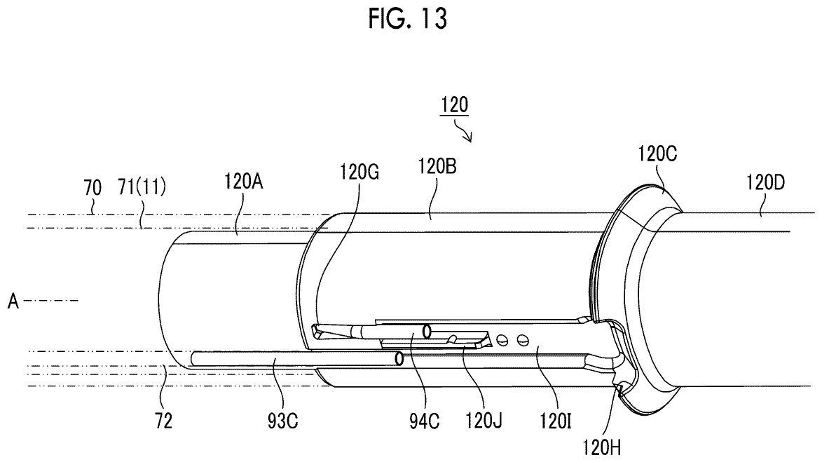

FIG. 13 is an enlarged view of a part of the second member 120 and the metal pipes 93C and 94C. As shown in FIG. 13, a stepped part 120I is formed along the central axis A direction on the outer peripheral surface of the second fitted portion 120B. The stepped part 120I is disposed at a position that is the same as a circumferential position with respect to the groove 120H. The stepped part 120I is a rectangular region recessed to the inner side (the side of the central axis A) compared to other portions of the second fitted portion 120B. The balloon air supply tube 93B and the liquid supply tube 94B are accommodated in the stepped part 120I. The stepped part 120I has an elongated portion corresponding to the shapes of the metal pipes 93C and 94C. Misregistration of the metal pipes 93C and 94C can be suppressed by the stepped part 120I.

A wall portion 120J formed along the central axis A direction is provided at a central portion of the stepped part 120I in the circumferential direction (a direction around the central axis A). The balloon air supply tube 93B and the liquid supply tube 94B are separated from each other and are positioned by the wall portion 120J.

The insertion assisting tool according to the embodiment of the present invention has been described in detail hereinbefore, but the present invention may include some improvements or modifications without departing from the scope of the present invention.

### EXPLANATION OF REFERENCES

1: endoscope device
   10: overtube
   11: insertion pipe line
   14: endoscope
   16: hand operating part
   18: insertion part
   20: universal cable
   21: connector
   24: light source device
   30: processor
   32: air/water supply button
   34: suction button
   36: shutter button
   38: angle knob
   39: forceps insertion part
   40: balloon
   40A: mounting part
   40B: mounting part
   40C: bulging part
   42: balloon air supply port
   44: soft portion
   46: bendable part
   48: tip part
   50: distal end surface
   52: observation window
   54: illumination window
   56: air and water supply nozzle
   58: forceps port
   60: monitor
   62: air supply and suction port
   70: overtube body
   71: first insertion pipe line
   72: air supply and discharge pipe line
   78: balloon
   80: gripping part
   80A: gripping part
   80B: gripping part
   81: second insertion pipe line
   82: insertion port
   84: first sleeve portion
   85: second sleeve portion
   86: insertion port forming portion
   87: flange portion
   87A: opening
   88: proximal end cap
   89: leakage prevention valve
   89A: opening
   90: outer peripheral surface
   90A: first surface
   90B: second surface
   90C: third surface
   90D: fourth surface
   93: balloon air supply pipe
   93A: balloon air supply port
   93B: balloon air supply tube
   93C: metal pipe
   94: liquid supply pipe
   94A: liquid supply port
   94B: liquid supply tube
   94C: metal pipe
   96: protrusion
   100: balloon control device
   102: hand switch
   104: tube
   106: tube
   110: first member
   110A: first body part
   110B: first flange portion
   110C: fitting passage
   120: second member
   120A: first fitted portion
   120B: second fitted portion
   120C: second flange portion
   120D: second body part
   120E: diameter increased portion
   120F: penetration passage
   120G: through-hole
   120H: groove
   120I: stepped part
   120J: wall portion
   840: first outer peripheral surface
   840A: side portion
   840B: arc portion
   840C: side portion
   840D: arc portion
   840E: connecting portion
   850: second outer peripheral surface
   850A: side portion
   850B: arc portion
   850C: side portion
   850D: arc portion
   850E: connecting portion
   860: outer peripheral surface
   860A: side portion
   860B: arc portion
   860C: side portion
   860D: arc portion
   860E: connecting portion
   A: central axis
   CE: curvature envelope
   H1: operator
   H2: assistant
   L1: distance
   L2: distance
   L3: distance
   L4: distance
   NV: normal vector
   C: circumscribed circle

What is claimed is:

1. An insertion assisting tool comprising:
a tube body that includes an insertion pipe line into which an endoscope insertion part is insertable and that is inserted into a body cavity; and
a tubular gripping part that is connected to a proximal end side of the tube body and that has an insertion port which communicates with the insertion pipe line,
wherein in cross-sectional view that is viewed from a direction orthogonal to an axial direction of the gripping part, at least a part of an outer peripheral surface of the gripping part has a shape formed by standardization.

2. The insertion assisting tool according to claim 1,
wherein in the outer peripheral surface of the gripping part, at least two surfaces facing each other in the direction orthogonal to the axial direction have the standardized shape.

3. The insertion assisting tool according to claim 2,
wherein in the outer peripheral surface of the gripping part, four surfaces facing each other in pairs in the direction orthogonal to the axial direction have the standardized shape.

4. The insertion assisting tool according to claim 1,
wherein the outer peripheral surface of the gripping part has a pair of side portions facing each other and a pair of arc portions connecting end parts of the pair of side portions to each other.

5. The insertion assisting tool according to claim 4,
wherein the side portion is an arc curve having a curvature radius larger than a curvature radius of the arc portion.

6. The insertion assisting tool according to claim 4,
wherein the side portion is a straight line.

7. The insertion assisting tool according to claim 4,
wherein a connecting portion between the side portion and the arc portion has a rounded shape without a corner portion.

8. The insertion assisting tool according to claim 4,
wherein the gripping part includes
a first sleeve portion that is provided on a side of the tube body, and
a second sleeve portion that is provided on a side of the first sleeve portion opposite to the side of the tube body and that has an outer shape smaller than an outer shape of the first sleeve portion, and
an outer peripheral surface of at least one of the first sleeve portion or the second sleeve portion has the pair of side portions and the pair of arc portions.

9. The insertion assisting tool according to claim 8,
wherein an outer peripheral surface of each of the first sleeve portion and the second sleeve portion has the pair of side portions and the pair of arc portions.

10. The insertion assisting tool according to claim 9,
wherein in cross-sectional view that is viewed from the direction orthogonal to the axial direction, the outer peripheral surface of the first sleeve portion and the outer peripheral surface of the second sleeve portion have shapes similar to each other.

11. The insertion assisting tool according to claim 8,
wherein the second sleeve portion has a length in the axial direction shorter than a length of the first sleeve portion in the axial direction.

12. The insertion assisting tool according to claim 8,
wherein the gripping part comprises an insertion port forming portion that is provided on a side of the second sleeve portion opposite to a side of the first sleeve portion, that has a funnel-like outer shape of which a diameter increases to the opposite side to the side of the first sleeve portion, and in which the insertion port is formed.

13. The insertion assisting tool according to claim 12,
wherein an outer peripheral surface of each of the first sleeve portion, the second sleeve portion, and the insertion port forming portion has the pair of side portions and the pair of arc portions.

14. The insertion assisting tool according to claim 13,
wherein in cross-sectional view that is viewed from the direction orthogonal to the axial direction, the outer peripheral surface of the first sleeve portion, the outer peripheral surface of the second sleeve portion, and the outer peripheral surface of the insertion port forming portion have shapes similar to each other.

15. The insertion assisting tool according to claim 8, further comprising:
a flange portion that has an outer shape larger than the first sleeve portion and the second sleeve portion, between the first sleeve portion and the second sleeve portion.

16. The insertion assisting tool according to claim 12,
wherein the insertion port forming portion comprises a leakage prevention valve that prevents a liquid which has passed through the insertion pipe line from flowing out from the insertion port.

17. The insertion assisting tool according to claim 1,
wherein in cross-sectional view that is viewed from the direction orthogonal to the axial direction, the outer peripheral surface of the gripping part has a shape of which curvature continuously changes along a circumferential direction.

18. The insertion assisting tool according to claim 17,
wherein the gripping part comprises
a first sleeve portion that is provided on a side of the tube body, and
a second sleeve portion that is provided on a side of the first sleeve portion opposite to the side of the tube body and that has an outer shape smaller than an outer shape of the first sleeve portion, and
an outer peripheral surface of at least one of the first sleeve portion or the second sleeve portion has a pair of side portions and a pair of arc portions.
